(19) United States
(12) Patent Application Publication
(10) Pub. No.: US 2021/0255358 A1
(43) Pub. Date: Aug. 19, 2021

(54) Method for estimating the subsurface total organic carbon (TOC) from well-log data

(71) Applicants: Manzar Fawad, Oslo (NO); MD Nazmul Haque Mondol, Oslo (NO)

(72) Inventors: Manzar Fawad, Oslo (NO); MD Nazmul Haque Mondol, Oslo (NO)

(21) Appl. No.: 17/175,749

(22) Filed: Feb. 15, 2021

Related U.S. Application Data
(60) Provisional application No. 62/978,344, filed on Feb. 19, 2020.

(51) Int. Cl. G01V 11/00; G01V 1/30; G01V 3/38; G01N 33/24

(52) U.S. Cl. CPC G01V 11/00; G01V 1/306; E21B 49/00; G01N 33/241; G01V 3/38

(57) ABSTRACT

The invention describes a procedure for determining the subsurface total organic content (TOC) from data obtained in the well by at least three well-logging tools measuring corresponding parameters. Three tools, namely sonic, density and deep resistivity are selected. The time interval signals from the sonic tool are converted to the P-wave velocity. The product of signals obtained from the sonic and density tools (P-wave velocity×Bulk density=Acoustic Impedance (AI)) responds in the same direction to a variation of the volume of water and organic matter (OM) volume of the rocks, whereas the third tool (Deep Resistivity) reacts very differently in response to a change of one or other of these same components, in a three-pole diagram, with rock matrix, OM and water as the three components onto an Acoustic Impedance vs resistivity ratio function plane. The resistivity ratio function is the square root of the ratio between the water resistivity and the measured formation resistivity. The position of the curved line with OM=0% by volume is fixed connecting the rock matrix pole with that of water pole. The slope of the matrix-water curve is controlled by the tortuosity factor ‘a’ that is a function of the rock pore structure, grain size and level of compaction. Iso-OM curves run parallel to this 0% OM reference curve. The data points to be analysed can be calibrated accordingly by changing the resistivity of water (Rw) and the tortuosity factor (a) parameters. In a graph where the parameters used depend, for example, on the sonic velocity in the rock, the rock bulk density and on the electric resistivity of the formations, the iso-OM lines form a set of parallel curved lines. The OM is derived from there corresponding to each pair of values of the parameters measured in the well. The obtained organic matter volume is converted to Total organic carbon (TOC) in gram percentage using a conventional relation.

### THE OBJECT OF THE INVENTION

The invention is a new method for estimating the total organic carbon (TOC) of the host-rocks from data recorded in bore-holes by well-logging tools.

The method of the invention focusses on the prediction of the TOC in percent of host-rocks by using data obtained from at least three logs of different kinds, a sonic time interval log, a bulk density log and a resistivity log. In the first case, a tool is lowered into a well containing sound wave transmitting and receiving transducers to measure the time interval of these waves propagating between the transmitter and the transducer through the rocks along the bore-hole penetration. In the second case, it is the density of rocks, whereas in the third case the electric resistivity of the formation surrounding the well is measured.

Estimation of the TOC of host-rocks is an important element in evaluating the hydrocarbon potential of a sedimentary basin. This invention generally relates to the field of hydrocarbon exploration, and more particularly to the characterisation of hydrocarbon (oil, gas, gas-condensates) source rocks in sedimentary basins (offshore and onshore) using the well-log data acquired in a borehole.

### BACKGROUND FOR THE INVENTION

The total organic matter content of sedimentary rocks is measured in a laboratory employing organic geochemistry methods. These analyses are run on well cuttings or core samples acquired in a well. The geochemical data obtained is patchy and incomplete, depending on the sample availability. The TOC information, therefore, is very often available in limited zones and absent where samples are not available, or the expensive laboratory analysis was not carried out. Issues of error in well-bore sample depths and contaminations may also result in inaccurate TOC representation. The estimation of TOC all along a well is therefore generally time-consuming, error-prone and expensive.

Researchers have come up with various methods linking the TOC to measurements made by well-logging tools of different types. Most of the methods are empirical and appropriate for a particular type of formation and area. The well logging technique comprises recording of magnitudes of various physical properties within a bore-hole using an array of logging probes lowered into the wellbore (FIG. 1).

Passey et al. (1990) came up with a method for petrophysical estimation of organic richness and a qualitative indicator of maturity called the Δ log R method, which uses the sonic log (as an indicator of porosity) and the deep resistivity log (Rd) as input (FIG. 2). The sonic curve is affected by the presence of low-density and -velocity kerogen, whereas the resistivity responds to the formation fluid. When these logs are overlain and appropriately scaled with a baseline in non-organic fine-grained lithology, a separation between the curves can be interpreted as effects related to organic-rich shales. If the source is immature, the separation is assumed to only be related to the sonic curve reacting to the properties of kerogen (slower transit time—indicating higher porosity). Where the source is matured, the resistivity increases as an additional response to hydrocarbons that have been generated (i.e. qualitative maturity indicator; FIG. 2). Sandstone hydrocarbon reservoirs with a similar resistivity response can generally be discriminated based on the gamma-ray signature.

The Δ log R value (separation between the curves) can then be measured at each depth interval. In order to transform Δ log R to TOC (in wt. %), an assumption about the source rock maturity in terms of LOM (Level of Organic Metamorphism units) is needed. It must either be determined through laboratory analysis (e.g. vitrinite reflectance or Rock-Eval pyrolysis) or estimated/guessed based on appearance of the resistivity log, burial history and temperature regime. For reference, LOM=7 corresponds to the onset of maturity for oil-prone kerogen, and LOM=12 corresponds to over-maturity.

Background organic content in non-source shales is neglected in the calculation of TOC. Organic matter type (kerogen type) generally do not influence the TOC predictions except in case of coals. Limitations for this method include feldspar rich sandstone reservoirs with high gamma-ray values, uncompacted sediments, tight intervals with low porosity and somewhat subjective calibration of the baseline values which can vary both from well to well and within the same well.

Another method for TOC estimation using the sonic and resistivity logs is Carbolog method (expired U.S. Pat. No. 5,126,939; Bessereau et al. 1991) that requires calibrating of wells where TOC measurements are available from laboratory analyses, and subsequently predicting TOC in other wells in the same sedimentary basin. The estimation is based on a theoretical rather than empirical equation which involves organic matter sonic transit time (Δt), matrix sonic transit time and slope of the 0% organic matter line (matrix-clay/water line; FIG. 3A-B). Components included in the method are organic matter, pore water, clays (containing water) and other minerals than clay denoted “matrix”. An increase in Δt combined with a decrease in resistivity (Rd) corresponds to an increase in the water or clay content. If Δt and resistivity both increase it is assumed to be an increase in OM content.

Vernik and Landis in 1996 came up with with a solution of measuring TOC by using the relation between TOC in weight percent and bulk density which appeared to provide reliable predictions in the studied wells compared to the TOC from core/cuttings. The input parameters required were the kerogen density that is dependent on maturity, the matrix density that in reality varies according to mineralogy and diagenesis, i.e., clay mineral transformation, the bulk density log measurement; and ‘k’, a constant related to the fraction of carbon in organic matter and can vary according to the maturation level. Earlier, a similar method from Schmoker (1979) was quite popular that used the bulk density tool measurements to calculate TOC. These approaches relied on signals from a single (density) tool.

Hakami et al. in 2016 used a modified equation (Schmoker & Hester 1983) that employs density information from wireline logs to calculate TOC. The study demonstrated a relationship between the acoustic impedance (AI) and TOC, whereby low AI correlates to high TOC values. However, AI as such was not utilized for TOC calculations.

### BRIEF SUMMARY

This method of the invention delivers improved results compared to the prior art, giving a continuous and representative estimation of the organic matter content of geological zones employing the data from well logging tools.

It is characterised in that it comprises: the use of data recorded by at least three well-logging tools measuring three different parameters, selected so that:
a) The product of measurements from tools one and two produce magnitude developing in the same direction in response to a volumetric change in the water, clay and organic matter content in sedimentary rocks,
b) the third tool yields measurement values ending up in opposite directions to each other in response to a volumetric increase in the organic matter content, on the one hand, and the water content, on the other, in the same host-rock, and
c) the three tool data are further selected so that the resulting pairs within the acoustic impedance-resistivity ratio plane correspond to an equal volume of organic matter content, associated respectively with sedimentary rocks comprising a given proportion of rock matrix or water, are substantially alike (in a graphic representation, these sets of pairs may be represented by iso-content curves), the plotting of collection of pairs of values corresponding to equal organic matter content volumes, so as to obtain a continuous depiction of the organic matter content of the rock zone penetrated by the well.

For example, the signals obtained from at least three well tools are used, adapted for determining the electric resistivity of the rock zone, the velocity of sound and the density through the same formation zone. A customised equation has been derived to solve for TOC volume using the information from the three said logs.

In a particular embodiment, measurements made by a well probe are used acquiring the electric resistivity of the ground and the other two tools measuring, for example, the velocity of sound and the density through this same ground, a representation diagram is chosen as a function of the resistivity ratio and of the Acoustic Impedance where said system of pairs of values of the properties measured may be likened to a system of substantially parallel iso-content curves, the organic matter content related with each pair of values of the Acoustic Impedance and that of resistivity ratio function acquired in the well then being determined by identifying the iso-content curve passing through the point characteristic of said pair in the selected representative diagram.

The method of the invention has following advantages compared to the prior art:

The complete and continuous information which it provides inexpensively using the existing wireline log data compared to the expensive prior art geochemical analysis carried out on a restricted number of samples. It is easy to use compared to the time consuming and subjective to interpretation Passey (1990) method that requires knowledge of Level of Organic Metamorphism units) in the area. It must either be determined through laboratory analysis (e.g. vitrinite reflectance or Rock-Eval pyrolysis) or estimated/guessed based on appearance of the resistivity log, burial history and temperature regime. Furthermore the Passey method utilizes data from two probes namely sonic and resistivity the curved iso-organic content lines are more accurate representation of the natural samples within the acoustic impedance-resistivity ratio domain utilizing data from three tools compared to the Carbolog method (expired U.S. Pat. No. 5,126,939; Bessereau et al. 1991) that made use of linear functions to create iso-content lines using information from two probes (sonic and resistivity). In the present invention the usage of resistivity ratio function on the Y-axis of the AI-resistivity ratio function plane provides a fixed standard template for the analysis; furthermore, it facilitates calibrating the data comprising the zero percent organic matter content with the reference zero percent organic matter curve just by iterating the Rw value.

The flexibility which it provides in terms of the number of parameters compared to the prior arts i.e. Schmoker (1979), Vernik & Landis (1996), Schmoker & Hester (1983), and Hakami et al. (2016) makes it usable in a wide range of geological environment. The signals from the sonic, density and resistivity tools that the present invention utilises provide a cushion from a possible minor error introduced by one of the tools. The problem aggravates where signals only from one tools are used as in case of the said prior art methods.

### BRIEF DESCRIPTION OF THE DRAWINGS

Other features and advantages of the invention will be better understood from the following detailed description and the attached drawings in which:

FIG. 1 illustrates typical wireline log data acquisition for subsurface sonic interval time, rock bulk density and resistivity determination;

FIG. 2 is an illustration for interpretation of Delta Log R responses using sonic—resistivity log overlay in various sub-surface formations, as disclosed in Passey et al.-supra;

FIG. 3 A-B depict the Carbolog method for TOC estimation with correlation of the clay, matrix, organic matter on the response of sonic and resistivity of the organic-rich rock (A) and organic matter contour lines on the response of sonic and resistivity of the organic rich rocks, as disclosed in Carpentier et al. supra and Bessereau et al (1991);

FIG. 4 shows a set of iso-organic matter content curved lines in a three-pole diagram onto AI—Resistivity ratio plane;

FIG. 5 illustrates the plotting of the set of pairs on the same diagram of values of the parameters acquired in a well by three well-logging probes before the Rw calibration;

FIG. 6 shows the plotting on the same diagram of the set of pairs of values of the parameters obtained in a well by three well-logging probes after the Rw calibration;

FIG. 7 depicts example of graph showing the similarity of the TOC values obtained by the present method of the invention and by the traditional method of geochemical analysis;

FIG. 8 A-B is another example of graph showing the TOC values estimated by the present inventive method and that of measured using conventional geochemical analysis (A), and a TOC distribution plot of the values obtained by the method of invention with quantities representing statistical output (B);

FIG. 9 is a flowchart showing elementary steps in one embodiment of the present inventive method.

### DETAILED EXAMPLES

The method of the invention comprises the use of data acquired by well-logging tools making it possible to separate the influence of organic matter and, thus, to estimate its weight percentage within sedimentary rocks.

Organic-rich rocks consist of three components: (1) the rock matrix (clay & quartz grains), (2) the Solid organic matter, and (3) the fluid(s) within the pore space (water or oil/gas). Non Source rocks are composed primarily of only two components: the matrix and the fluid filling the pore space. In immature source rocks, organic matter and rock matrix make up the solid fraction, and formation water fills the pore space. As the source rock matures, a part of the solid organic matter is converted to liquid (or gaseous) hydrocarbons that occupy the kerogen/organic matter pore space.

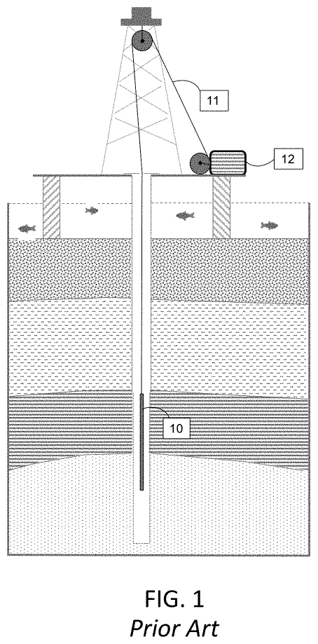

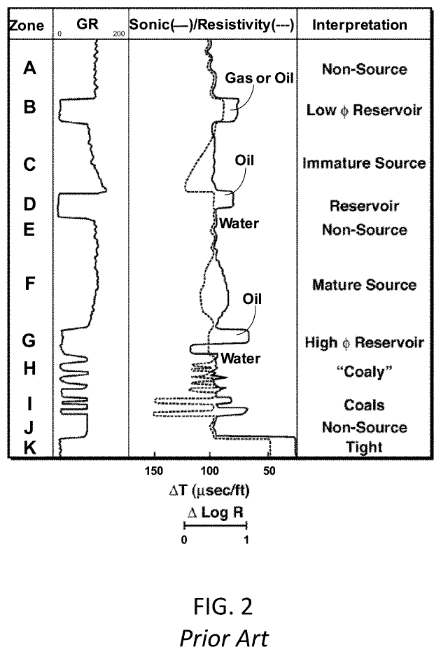

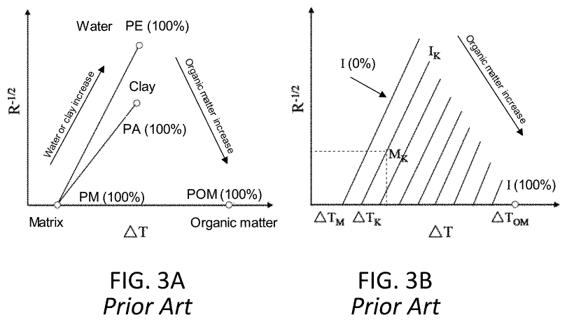

The method of the invention makes use of data acquired from a wellbores drilled through subsurface rock formations in an area of interest. Data obtained from the wellbore may include so called “well log” data. Such data are typically recorded and presented with respect to depth in the subsurface of various physical parameters measured by probes lowered into the wellbore. Such probes may include, for example, electrical resistivity, acoustic interval time, bulk density, neutron slowing down length, neutron capture cross section, natural gamma radiation, and nuclear magnetic resonance relaxation time distribution, among others. The well logging procedure comprises recording of magnitudes of various above mentioned physical properties within a bore-hole using an array of logging probes (FIG. 1, 10), attached with a logging cable (11) connected on the other end to a data recording cabin (12).

The method of the invention contains first of all obtaining the well log data and the selection of three well-logging probes appropriate for predicting the magnitude of organic matter. The response of well-logging tools is dependent on the properties related to the components as well as their respective percentage in the rocks investigated. The tool measuring the sonic transit time through the formations is sensitive to the water, organic matter and volume of matrix content. The sonic interval time is converted to sonic velocity. The probe measuring the density is sensitive to water and to the organic matter and the void spaces/porosity between the matrix grains. The tool that measures the electric resistivity of the rock makes slight discrimination between the wet clay and the saline water as both are conducting agents, and no discrimination for variations in composition of the matrix if the conducting minerals are not in a continuous phase. The product of density with sonic derived velocity is called acoustic impedance. We used acoustic impedance values as a combined augmented response of the sonic and density probes within the method of invention. A function namely resistivity ratio function was introduced within the method of invention. The resistivity ratio function was defined as the square root of the ratio between the resistivity of Formation water and the resistivity measured by the resistivity tool.

In low-TOC water-wet porous rocks, the two curves i.e. acoustic impedance and resistivity ratio respond to porosity. But in high-TOC source rocks both the acoustic impedance and resistivity curves respond due to two main effects: 1) the acoustic impedance curve responds to the presence of low-density low-velocity kerogen, and 2) the resistivity ratio curve responds to the porosity and formation fluid. When maturity in a high-TOC source rocks is low and no hydrocarbons have been generated, both the acoustic impedance and resistivity ratio response is caused only by the porosity response to low density and/or low velocity TOC. Conversely, when maturity is high in such organic-rich rocks, the resistivity response increases due to the generated hydrocarbons. Since the generated hydrocarbon stay within the pores of organic matter (Alfred & Vernik 2013), assumption to include this hydrocarbon volume with the organic matter, and considering the porosity equal to the volume only filled by water simplifies the process of isolating the organic matter volume. In an organic rich rock 100% matrix content with zero porosity, or 100% organic matter with zero porosity is assumed to yield infinity resistivity, resulting in zero resistivity ratio values. On the other hand at water pole the resistivity of water (R_{w}) theoretically becomes equal to the total resistivity (Rₜ) resulting in resistivity ratio value of 1.

The two properties obtained from the well log data are chosen also so that the collection of pairs of values of acquired parameters (namely the acoustic impedance on the one hand and the resistivity ratio function on the other) at least partly correspond to the equal organic matter content volume for sedimentary rocks comprising a given proportion of matrix or water are substantially identical.

This selection of petrophysical parameters considerably simplifies the operation for estimating the organic matter content. In a cross-plot of the two chosen properties, the collection of pairs of values of the said parameters are spread over iso-organic matter content curves. A diagram may be drawn where the iso-organic matter curved lines are parallel to the reference curved line representing 0% (or 0 fraction) organic matter which joins the 100% (or 1 fraction) water pole to the 100% (or 1 fraction) matrix pole.

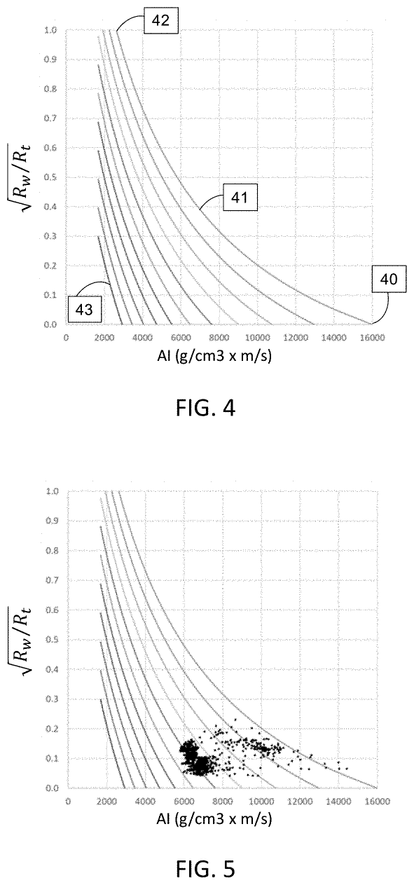

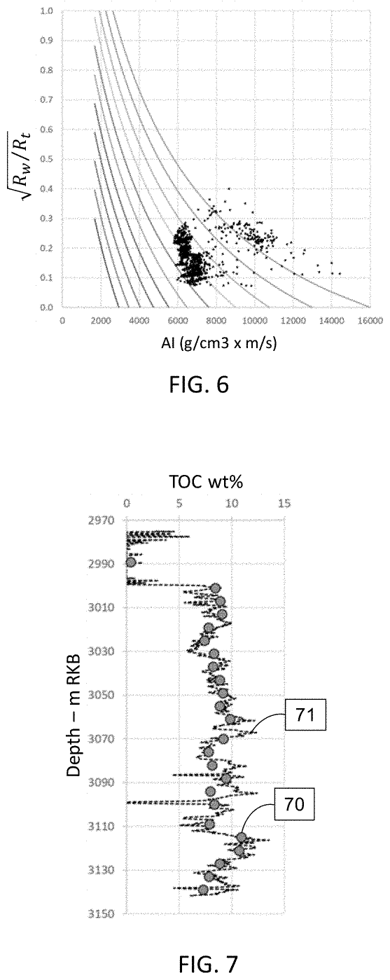

The baseline represented by the X-axis along the resistivity ratio function (\sqrt{R_{w}/R_{T}})=0 was assumed to be having infinity resistivity and zero porosity. If we assume the rock consists of matrix, organic matter (OM) and water-filled matrix porosity then collection of pairs of values of the parameters serving as reference which is represented by the iso-content curved line equivalent to a given organic matter percentage within a rock obtained experimentally from values of the two chosen parameters acquired along a well. In the diagram as a function of AI and \sqrt{R_{w}/R_{T}} this reference line is the curved reference line with 0% organic content volume (FIG. 4, 41) extending from the 100% (or 1 fraction) matrix pole (40) joining the 100% (or 1 fraction) water pole (42).

This method of determining the R_{w} to align the 0% organic matter zone data along the 0% organic matter reference line implies that, among the zones crossed by the well, some is devoid of organic matter. This is possible if we assume the data pairs with lowest resistivity ratio function values occasionally showing a trend partly parallel to the 0% organic matter reference line. It is possible to verify the existence of such zones by comparison with geochemical analysis results within a basin.

The pairs of values are represented by the set of iso-content curved lines, from the line with 0% (or 0 fraction) organic content (41) to the line representing 100% (or 1 fraction) volumetric organic matter content (43). From the following function (equation 1) we are able to define a set of lines corresponding to different organic matter volumes (fraction), joining different matrix-OM ratios with the water point in the resistivity ratio function-Acoustic impedance plane (FIG. 4):

$$\sqrt{\frac{{R}_{w}}{{R}_{t}}} = \frac{{ρ}_{m a} + {V}_{o m} [ ( {ρ}_{o m} - {ρ}_{m a} ) - AI ( \frac{1}{{V}_{{p}_{o m}}} - \frac{1}{{V}_{{p}_{m a}}} ) ] - \frac{A I}{{V}_{{p}_{m a}}}}{\sqrt{a} [ AI ( \frac{1}{{V}_{{p}_{o m}}} - \frac{1}{{V}_{{p}_{m a}}} ) - ( {ρ}_{w} - {ρ}_{m a} ) ]} \qquad ( 1 )$$

where V_{Pma}, V_{Pom} and V_{Pw} are the P-wave velocities of the mineral matrix, organic matter (OM) and the pore fluid (water) respectively, ρₘₐ is density of mineral grains, porn is density of organic matter (OM), ρ_{w} is density of pore fluids that is water in this case, Rₜ is deep resistivity, R_{w} is the resistivity of water, ‘a’ is tortuosity factor, AI is acoustic impedance and Vₒₘ is the volume of organic matter in fraction. The tortuosity factor ‘a’ controls the slope of the iso-content curved lines and may be selected in a formation zone depending on pore structure, grain size and level of compaction. The relevant constants may be taken from Mavko et al (2009) and vendors' logging chart books.

Rearranging the equation the volume of organic matter can be calculated in volume percent:

$${V}_{o m} = \frac{\frac{A I}{{V}_{{p}_{m a}}} {ρ}_{m a} + \sqrt{\frac{a {R}_{w}}{{R}_{t}}} [ AI ( \frac{1}{{V}_{{p}_{w}}} - \frac{1}{{V}_{{p}_{m a}}} ) - ( {ρ}_{w} - {ρ}_{m a} ) ]}{[ ( {ρ}_{o m} - {ρ}_{m a} ) - AI ( \frac{1}{{V}_{{p}_{w}}} - \frac{1}{{V}_{{p}_{m a}}} ) ]} \times 100 \qquad ( 2 )$$

Since the R_{w} is unknown, iterate the value of R_{w} make the upper right part of the data representing the matrix to fall on the 0% OM line (FIGS. 5&6). This R_{w} is not the actual resistivity of water, but rather an apparent R_{w} that compensates for the deviation from initial assumptions since the actual rock consists of wet clays which are good conductors. The rock's effective resistivity responds to a more complex function depending on the porosity, R_{w}, volume of shale (Vₛₕ) and resistivity of shale (Rₛₕ) (Bessereau et al. 1991).

Finally, the volumetric contents obtained Vₒₘ(%) are transformed into TOC in weight percentage using the relation:

$$TOC = {V}_{o m} \frac{{ρ}_{o m}}{{ρ}_{b}} \frac{1}{k} \qquad ( 3 )$$

where TOC is organic carbon content (weight %), Vₒₘ is volume % of OM, ρₒₘ is the density of the organic matter, Pb is the bulk density of the rock and ‘k’ is a ratio between the organic carbon and the organic matter. The factor ‘k’ is dependent on the type and level of maturation of organic matter (Tissot & Welte 2013). This transformation facilitates comparisons with the results obtained by geochemical analyses. The bulk density (ρ_{b}) used in the relation (3) may be obtained employing the probe measuring the density in the well.

In the case where, geochemical analysis is available from the well, a comparison may be made between it and the logging data to obtain an average value of the resistivity for formation water ‘R_{w}’, the tortuosity factor ‘a’ and the conversion factor ‘k’ subsequently to apply on the other wells data.

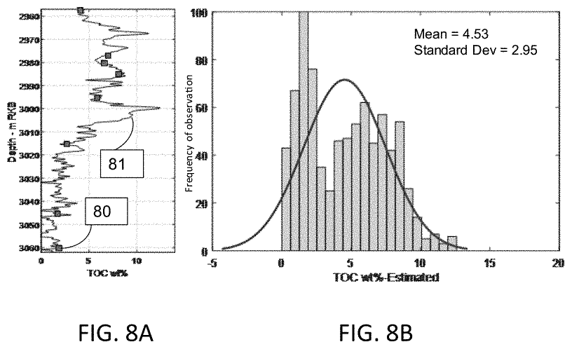

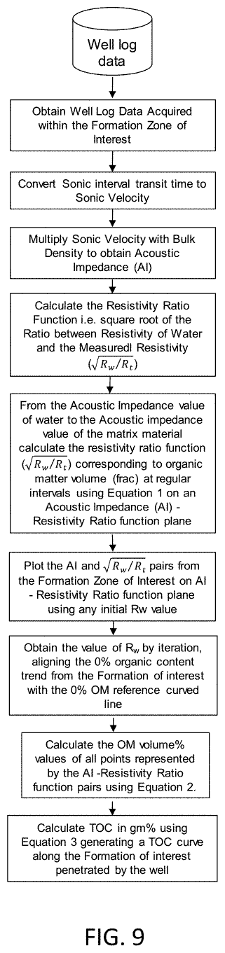

It has been substantiated experimentally that the results obtained by the method of the invention agreed well with those where the TOC from geochemical analysis was available, as shown by the examples in FIGS. 7 and 8A. In these figures, the scatter points are obtained from analyzed cuttings samples (70 & 80), whereas the variation of the TOC estimated from the present invention is represented by a dotted and a continuous curve, respectively (71 & 81). A statistical distribution of the TOC may be obtained from the obtained TOC % continuous curve (FIG. 8B). The procedure workflow which is followed so as to obtain the plot of the TOC % against depth is shown in FIG. 9.

The resistivity of water (Rw), tortuosity factor (a) and the conversion factor (k), which are functions of rock depositional environment, mineralogy, organic matter type, and maturity may vary in nature within the same area. A stochastic approach employing Monte Carlo simulations can be utilised to take into account the resultant TOC uncertainty. The input values of Rw, a, and k, in this case, will be fed randomly in the form of normal, or other suitable distributions.

The technical solution is only one embodiment of the present invention, to those skilled in the art, the present invention discloses a fundamental principle of the method and applications, straightforward to make various types of modifications or variations, the method is not limited to the specific embodiments of the present invention described above, and therefore the manner described above are only preferred and is not in a limiting sense.

### REFERENCES CITED

PATENT DOCUMENTS

| | | |
|---|---|---|
| U.S. Pat. No. 5,126,939 | June 1992 | Carpentier and Huc |

### PUBLICATIONS

0039 Alfred, D. & L. Vernik (2013): “A new petrophysical model for organic shales”, Petrophysics, 54, 03, 240-247.
Bessereau, G., B. Carpentier & A. Y. Huc (1991): “Wireline Logging And Source Rocks-Estimation Of Organic Carbon Content By The Carbolog Method”, Log Anal., 32, 03.
Hakami, A., A. Al-Mubarak, K. Al-Ramadan, C. Kurison, & I. Leyva (2016): “Characterization of carbonate mudrocks of the Jurassic Tuwaiq Mountain Formation, Jafurah basin, Saudi Arabia: Implications for unconventional reservoir potential evaluation”, Jour. of Natural Gas Science and Engineering, 33, 1149-1168.
Mavko, G., T. Mukerji & J. Dvorkin (2009): The rock physics handbook: Tools for seismic analysis of porous media, Cambridge university press.
Passey, Q. R., S. Creaney, J. B. Kulla, F. J. Moretti & J. D. Stroud (1990): “A practical model for organic richness from porosity and resistivity logs”, AAPG Bull., 74, 12, 1777-1794.
Schmoker, J. W. (1979):—“Determination of organic content of Appalachian Devonian shales from formation-density logs: Geologic notes”, AAPG Bull., 63, 9, 1504-1509.
Schmoker, J. W., & T. C. Hester (1983): “Organic carbon in Bakken formation, United States portion of Williston basin”, AAPG Bull., 67, 12, 2165-2174.
Tissot, B. P. & D. H. Welte (2013): Petroleum formation and occurrence, Springer Science & Business Media.
Vernik, L. & C. Landis (1996): “Elastic anisotropy of source rocks: Implications for hydrocarbon generation and primary migration”, AAPG Bull., 80, 4, 531-544.

The invention claimed is:

1. A method for quantifying the total organic carbon of sedimentary rocks within a sedimentary basin using well-logging data measured in a well, comprising:
using data provided by at least three well-logging probes measuring three different parameters selected so that:
a) The product of the velocity of sound obtained from one tool with the density data obtained from the second tool, hereby called acoustic impedance develop in the same direction in response to a volumetric change of the water, clay and organic matter content in the said sedimentary rocks,
characterised by
b) the third probe produces measurement signals hereby modified to a resistivity ratio function developing in opposite directions to each other due to the organic matter content variation, on the one hand, and the water content, on the other, in the same sedimentary rocks, and
c) the three probes being further selected so that the resulting pairs within the acoustic impedance-resistivity ratio plane correspond to an equal organic matter content, associated respectively with the said rocks comprising a given percentage of rock matrix or water, are equal represented by one pair of values of the representative parameters of the pure organic matter, creating a system of sets of pairs of values of the acquired parameters, to obtain a continuous representation of the volumetric organic matter content of the formations penetrated by the well,
using equation
$$d ) {V}_{o m} = \frac{\frac{A I}{{V}_{{p}_{m a}}} - {ρ}_{m a} + \sqrt{\frac{a {R}_{w}}{{R}_{t}}} [ AI ( \frac{1}{{V}_{{p}_{w}}} - \frac{1}{{V}_{{p}_{m a}}} ) - ( {ρ}_{w} - {ρ}_{m a} ) ]}{[ ( {ρ}_{o m} - {ρ}_{m a} ) - AI ( \frac{1}{{V}_{{p}_{om}}} - \frac{1}{{V}_{{p}_{m a}}} ) ]} \times 100$$
where V_{Pma}, V_{Pom}, and V_{Pw} are the P-wave velocities of the mineral matrix, organic matter (OM) and the pore fluid (water) respectively, ρₘₐ is the density of mineral grains, ρₒₘ is the density of organic matter (OM), ρ_{w} is the density of pore fluids that is water in this case, Rₜ is deep resistivity, R_{w} is the resistivity of water, ‘a’ is tortuosity factor, AI is acoustic impedance, and Vₒₘ is the volume of organic matter in percent.

2. The method of claim 1, wherein the measurements made by at least three well probes are employed, adapted for measuring the electric resistivity of the formation penetrated, the transit time of sound through the same ground, and the density of the said ground.

3. The method as claimed in claim 2, a resistivity ratio function is defined as the square root of the ratio between the resistivity of water and the resistivity values obtained from the resistivity probe.

4. The method of claim 2, wherein measurements made by a well probe measuring the electric resistivity of the zone in the sub-surface and two other well probes measuring the transit time of sound and the density through this same zone, a representation diagram is chosen as a function of the resistivity ratio function and of the acoustic impedance where said system of sets of pairs of values of the parameters acquired, each associated with the same content, may be likened to a set of parallel iso-content curves, the organic matter content associated with each pair of values of the acoustic impedance and of the resistivity ratio measured in the well then being determined by identifying the iso-volumetric content curve passing through the point representative of said pair in the chosen representation diagram.

5. The method of claim 2, wherein the slope of iso-volumetric content curves is controlled by the tortuosity factor ‘a’ that is selected for a formation zone considering the pore structure, grain size and level of compaction.

6. The method of claim 2, wherein the resistivity of water is determined by iterating the resistivity of water while aligning the 100% water-saturated well data onto the acoustic impedance—resistivity ratio plane with the 0% fluid saturation reference curved line.

7. The method of claim 2, wherein measurements are used made by a well probe measuring the electric resistivity of the ground, and two other probes, one measuring the speed of sound within the ground and the other density.

8. The method of claim 1, wherein the weight percentage of total organic carbon are further determined associated respectively with the values of the volumetric contents obtained.

9. The method of claim 1, wherein the reference set is established by selecting, from all the pairs of values acquired in the well, at least one specific pair of quantities for which a given organic matter percentage volume may be associated.

10. The method of claim 1, wherein quantities from each pair of the parameters acquired in the well is demonstrated in a diagram as a function of coordinates, one measuring acoustic impedance in the rock and the other the square root of the ratio between the resistivity of water and the resistivity of rock, hereby called the resistivity ratio function, where the collection of pairs of values equivalent to a corresponding content are manifested by a system of curved lines parallel to a reference curved line representing a zero percent organic volume content, to which a given organic matter volume percentage may be allocated.

11. The method of claim 11, wherein the positions of the iso-volumetric content curved lines are determined between an axis with the 100% rock matrix member on one end and the 100% organic matter on the other end, both represented by the values taken by the two parameters.

12. The method of claim 1, wherein the pair of values typical of the pure organic matter, pure matrix and water are obtained from the existing literature.

13. The method of claim 1, wherein the obtained results of the total organic carbon from a zone of formation represent the statistical distribution within the zone investigated.

14. The method of claim 1, wherein to obtain stochastic TOC results the distribution of input parameters are to be fed in random manner performing calculations using Monte-Carlo simulation.

15. The method of claim 1, wherein resistivity and acoustic impedance obtained from other subsurface methods may be used to estimate the TOC of rocks in the subsurface.
